(19) United States
(12) Patent Application Publication
(10) Pub. No.: US 2024/0062676 A1
(43) Pub. Date: Feb. 22, 2024

(54) DIGITAL CONTACT LENS INSERTION AND REMOVAL AID

(71) Applicant: Johnson & Johnson Vision Care, Inc., Jacksonville, FL (US)

(72) Inventors: Kurt Moody, Jacksonville, FL (US); Charissa Lee, Jacksonville, FL (US); Frederick Williams, Jacksonville, FL (US)

(21) Appl. No.: 17/891,736

(22) Filed: Aug. 19, 2022

(51) Int. Cl. G09B 19/00

(52) U.S. Cl. CPC G09B 19/003; G06F 3/0482

(57) ABSTRACT

Disclosed herein are methods for methods, systems, and user interfaces for contact lens insertion and removal. An example method may comprise receiving one or more images of at least a portion of a user. The one or more images may comprise a representation of an eye of the user and a representation of a contact lens on the eye of the user. The example method may comprise analyzing, based on the one or more images, placement of the contact lens on the eye of the user. The example method may comprise outputting an indication of proper placement or improper placement of the contact lens based on the analyzing placement of the contact lens.

### BACKGROUND

Patients new to soft contact lenses often struggle with insertion and removal of their contact lenses. This frustration can lead to frustration, discomfort and/or discontinuation of lens wear. Often the patient may be successful when being taught the tasks contact insertion and removal in the office of an eye-care professional (ECP) but then cannot replicate the tasks once the patient is home. In the office, the patient has the advantage of getting feedback from an office staff member, but, when home attempting to insert or remove the lens, the patient does not have that advantage.

Improvements are needed.

### SUMMARY

Disclosed herein are methods, systems, and user interfaces for contact lens insertion and removal that aid in helping the novice soft contact lens patient insert, remove, and alert them when the contact lens was correctly on the eye. If patients had a tool that would provide them with feedback on insertion and removal of a lens outside of the eye care providers office, then contact lens drop out may be reduced.

The present disclosure relates to a digital (e.g., app) solution that may be considered a virtual ophthalmic technician that may aid in helping the novice soft contact lens patient insert and remove a contact lens and alerting the patient when the contact lens is correctly on the eye. Controlling upper and lower eye lids correctly are key to insertion and removal thus the methods, systems, and user interfaces disclosed herein may utilize a camera in a device such as a mobile phone or tablet to project visual clues on upper and lower eye lids to assist a patient in correctly controlling eye lids with the patient's fingers during insertion and removal. When the fingers are in the correct position, the visual clues may turn confirmatory color, such as green. When the fingers are not in the correct position, the visual clues may turn red, for example. The methods, systems, and user interfaces disclosed herein may advise the patient step by step via voice or sound control to successfully insert and remove contact lenses.

One aspect to successful insertion and removal may be knowing the contact lens is on the eye in the proper position. Thus, it may be useful for the methods, systems, and user interfaces disclosed herein to be able to detect a contact lens being centrally resident on eye. To determine if the contact lens was inserted correctly, the methods, systems, and user interfaces disclosed herein may leverage edge detection technology to identify correct placement. The methods, systems, and user interfaces disclosed herein may be used in a home setting to reinforce the in-office teaching of contact lens insertion and removal when office staff is not available. Also eye care specialists and staff may use the methods, systems, and user interfaces disclosed herein to help with in-office training.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for contact lens assistance. The method also includes causing, via a user interface, output of one or more lens type options; receiving, via the user interface, an indication of a select lens type of the one or more lens type options; causing, via the user interface, output of one or more lens operation options; receiving, via the user interface, an indication of a select lens operation of the one or more lens operation options; causing, via the user interface, output of one or more eye options; receiving, via the user interface, an indication of a select eye of the one or more eye options; causing, via the user interface and based upon one or more of the select lens type, the select lens operation, or the select eye, output of one or more user instructions for insert or removal of a contact lens, where the one or more user instructions incorporate a user feedback based on images of the user captured in real time. Other embodiments of this aspect include corresponding computer systems, apparatus, and computer programs recorded on one or more computer storage devices, each configured to perform the actions of the methods.

One general aspect includes a method for contact lens assistance. The method also includes receiving one or more first images of at least a portion of a user, where the one or more first images may include a representation of an eye of the user; causing, via a user interface and based on the one or more first images, output of a visual clue indicative of a user state of the user relative to the eye of the user, where the visual clue has a first state indicative of a rejected user state and a second state indicative of an accepted user state; causing, in response to an accepted user state, output of one or more user instructions for insertion of a contact lens; receiving one or more second images of at least a portion of a user, where the one or more second images may include a representation of the eye of the user and a representation of a contact lens on the eye of the user; analyzing, based on the one or more second images, placement of the contact lens on the eye of the user; and outputting an indication of proper placement or improper placement of the contact lens based on the analyzing placement of the contact lens. Other embodiments of this aspect include corresponding computer systems, apparatus, and computer programs recorded on one or more computer storage devices, each configured to perform the actions of the methods.

One general aspect includes a method for contact lens assistance. The method also includes receiving one or more images of at least a portion of a user, where the one or more images may include a representation of an eye of the user and a representation of a contact lens on the eye of the user; analyzing, based on the one or more images, placement of the contact lens on the eye of the user; and outputting an indication of proper placement or improper placement of the contact lens based on the analyzing placement of the contact lens. Other embodiments of this aspect include corresponding computer systems, apparatus, and computer programs recorded on one or more computer storage devices, each configured to perform the actions of the methods.

One general aspect includes a method for contact lens assistance. The method also includes receiving a real-time video of at least a portion of a user, where the video may include a representation of an eye of the user; analyzing, based at least on the video, a user state of the user to determine whether the user state is an accepted user state or a rejected user state; causing, via a user interface and based on the video, output of a visual clue indicative of the user state of the user relative to the eye of the user, where the visual clue has a first state indicative of a rejected user state and a second state indicative of an accepted user state; causing, via a user interface and based on the video and based on the user state, output one or more real-time prompts to be outputted to the user; and repeating steps a-d until the user state is determined to be an accepted user state. Other embodiments of this aspect include corresponding computer systems, apparatus, and computer programs recorded on one or more computer storage devices, each configured to perform the actions of the methods.

### BRIEF DESCRIPTION OF THE DRAWINGS

The following drawings show generally, by way of example, but not by way of limitation, various examples discussed in the present disclosure. In the drawings:

FIG. 1 depicts an example flowchart for a method in accordance with the present disclosure.

FIG. 2 depicts example user interface screens in accordance with the present disclosure.

FIG. 3 depicts an example flowchart for a method in accordance with the present disclosure.

FIG. 4 depicts an example flowchart for a method in accordance with the present disclosure.

FIG. 5 depicts an example flowchart for a method in accordance with the present disclosure.

FIG. 6 depicts an example image of a user's eye in accordance with the present disclosure.

FIG. 7 depicts the example image of FIG. 6 with overlaid calculated boundaries.

FIG. 8 depicts an example masked image after implementing an intelligent masking technique over the image of FIG. 6 to remove image data outside of a defined image window in accordance with the present disclosure.

FIG. 9 depicts the masked image of FIG. 8 showing possible boundary arcs in accordance with the present disclosure.

FIG. 10 depicts the masked image of FIG. 8 showing representative circles for identifying correct and false representative circles in accordance with the present disclosure.

### DETAILED DESCRIPTION

The present disclosure relates to methods, systems, and user interfaces for contact lens insertion and removal. A user may use a camera of a smart device to record video of the user attempting to insert and/or remove a contact lens. A screen of the smart device may provide the user with instant feedback. The instant feedback may comprise approval and/or disapproval of placement of the user's fingers, positioning of eyelids, positioning of a contact lens, etc. The methods, systems, and user interfaces described herein may reduce time, effort, and frustration associated with learning to insert and/or remove contact lenses.

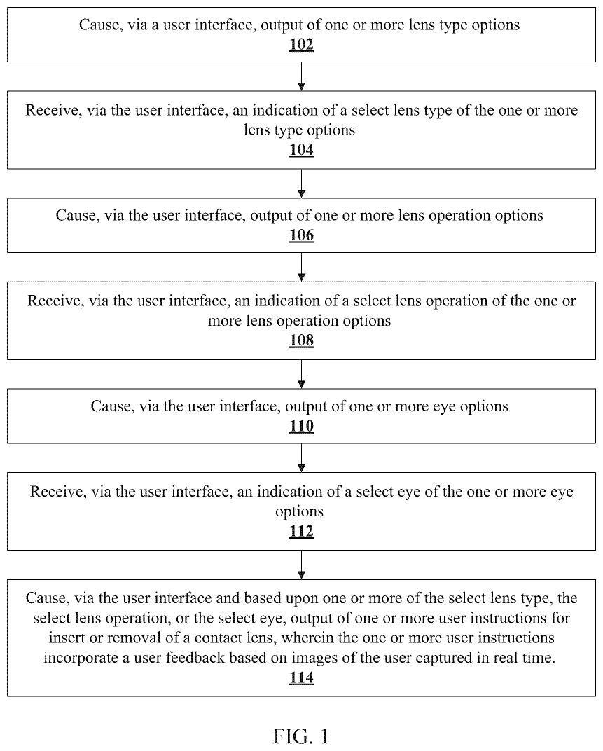

FIG. 1 depicts an example flowchart for a method for contact lens assistance such as insertion or removal of a contact lens from a user's eye(s). A system of one or more computers can be configured to perform particular operations or actions by virtue of having software, firmware, hardware, or a combination of them installed on the system that in operation causes or cause the system to perform the actions. As an example, a smartphone may be equipped to capture one or more images of a user (e.g., video, real-time video). As a further example, a user may implement the method on a device configured to display information to a user while capture “selfie” view images (e.g., video, real-time video) of the user.

At step 102, one or more lens type options may be caused to be outputted via a user interface. The user interface may be on any device or display. As an example, the user interface may be on a device configured to capture images of the face of the user while viewing the user interface. The lens type options may comprise a representation of one or more of reusable or daily disposable.

At step 104, an indication of a select lens type of the one or more lens type options may be received via the user interface. The user interface may be on any input device or touchable display. As an example, the user interface may be on a device configured to display options and receive an indication of selection of one of the displayed options. Receiving an indication of a select lens type of the one or more lens type options may comprise receiving an indication of engagement of a clickable element, such as a button, associated with the select lens type.

At step 106, one or more lens operation options may be caused to be outputted via the user interface. The user interface may be on any device or display. As an example, the user interface may be on a device configured to capture images of the face of the user while viewing the user interface. The one or more lens operation options may comprise a representation of one or more of insert or removal of a contact lens.

At step 108, receiving, via the user interface, an indication of a select lens operation of the one or more lens operation options. The user interface may be on any input device or touchable display. As an example, the user interface may be on a device configured to display options and receive an indication of selection of one of the displayed options. Receiving an indication of a select lens operation of the one or more lens operation options may comprise receiving an indication of engagement of a clickable element, such as a button, associated with the select lens operation.

At step 110, one or more eye options may be caused to be outputted via the user interface. The user interface may be on any device or display. As an example, the user interface may be on a device configured to capture images of the face of the user while viewing the user interface. The one or more eye options may comprise a representation of one or more of left eye or right eye.

At step 112, receiving, via the user interface, an indication of a select eye of the one or more eye options. The user interface may be on any input device or touchable display. As an example, the user interface may be on a device configured to display options and receive an indication of selection of one of the displayed options. Receiving an indication of a select eye of the one or more eye options may comprise receiving an indication of engagement of a clickable element, such as a button, associated with the select eye.

At step 114, one or more user instructions for insert or removal of a contact lens may be caused to be outputted via the user interface and may be based upon one or more of the select lens type, the select lens operation, or the select eye. The one or more user instructions may incorporate a user feedback based on images of the user captured in real time. The user interface may be on any device or display. As an example, the user interface may be on a device configured to capture images of the face of the user while viewing the user interface. The causing output of one or more user instructions for insert or removal of a contact lens may be executed for the select eye. One or more user instructions for insert or removal of a contact lens for an unselected eye of the one or more eye options may be automatically caused to be outputted via the user interface and based upon one or more of the select lens type or the select lens operation. The one or more user instructions may comprise wash and dry instructions. The one or more user instructions may comprise contact lens handling instructions. The one or more user instructions may comprise contact lens position instructions. The one or more user instructions may comprise contact lens orientation instructions. The one or more user instructions may comprise finger position instructions. The one or more user instructions may comprise camera configuration instructions. The one or more user instructions may comprise user feedback queries.

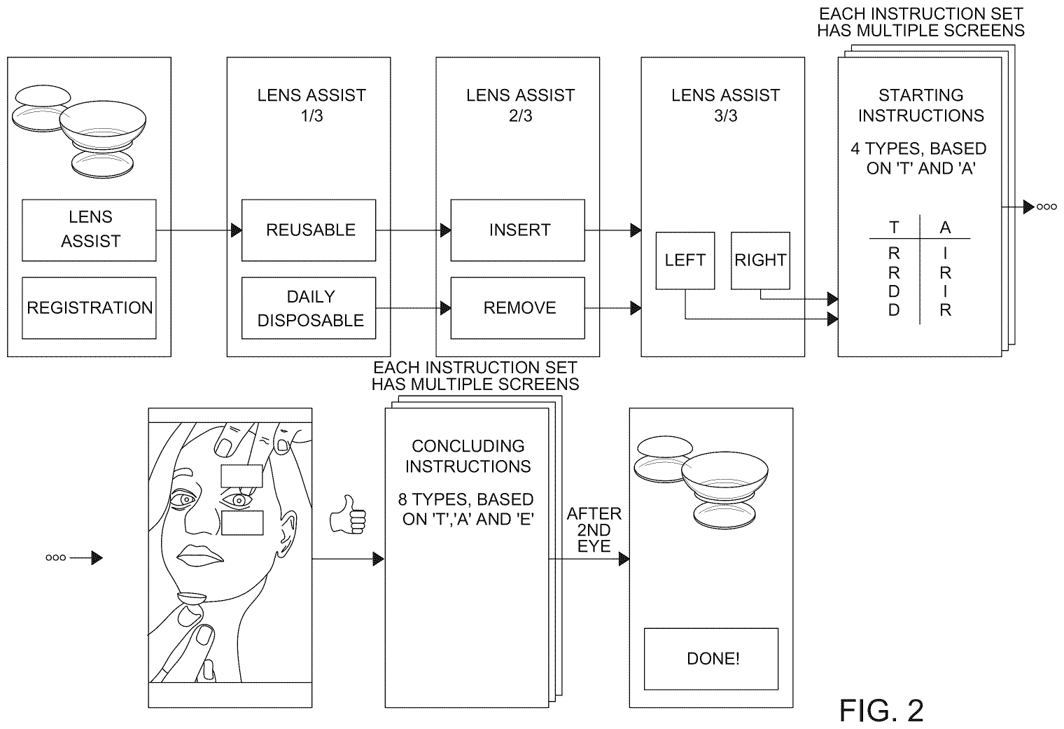

FIG. 2 illustrates example method flow for contact lens assistance such as insertion or removal of a contact lens from a user's eye(s). A system of one or more computers can be configured to perform particular operations or actions by virtue of having software, firmware, hardware, or a combination of them installed on the system that in operation causes or cause the system to perform the actions. As an example, a smartphone may be equipped to capture one or more images of a user (e.g., video, real-time video). As a further example, a user may implement the method on a device configured to display information to a user while capture “selfie” view images (e.g., video, real-time video) of the user.

The example method flow comprises an introductory (e.g., start, landing, etc.) page. The introductory page may comprise a button to launch one or more methods described herein. The introductory page may comprise a button to launch a registration procedure. Engagement of the button to launch one or more methods described herein may cause a first screen with lens type options to be displayed. The lens type options may comprise a reusable option and a daily disposable option. Each lens type option may be associated with a button on the first screen. Engagement of a button on the first screen may cause the associated lens type option to be selected and cause a second screen with lens operation options to be displayed. The lens operation options may comprise an insert option and a remove option. Each lens operation option may be associated with a button on the second screen. Engagement of a button on the second screen may cause the associated lens operation option to be selected and cause a third screen with eye options to be displayed. The eye options may comprise a left option and a right option. Each eye option may be associated with a button on the third screen. Engagement of a button on the third screen may cause the associated eye option to be selected and cause a set of instruction screens to be launched based on the selected options. The set of instruction screens may comprise written instructions. The set of instruction screens may comprise video of the user. The video of the user may comprise an overlay. The overlay may comprise indications of approval or disapproval of positioning of fingers, eyelids, contact lenses, etc. The set of instruction screens may comprise starting instructions. The set of instruction screens may comprise concluding instructions. On conclusion of the instructions for the selected eye, the set of instructions may comprise instructions relevant to an eye that was not selected.

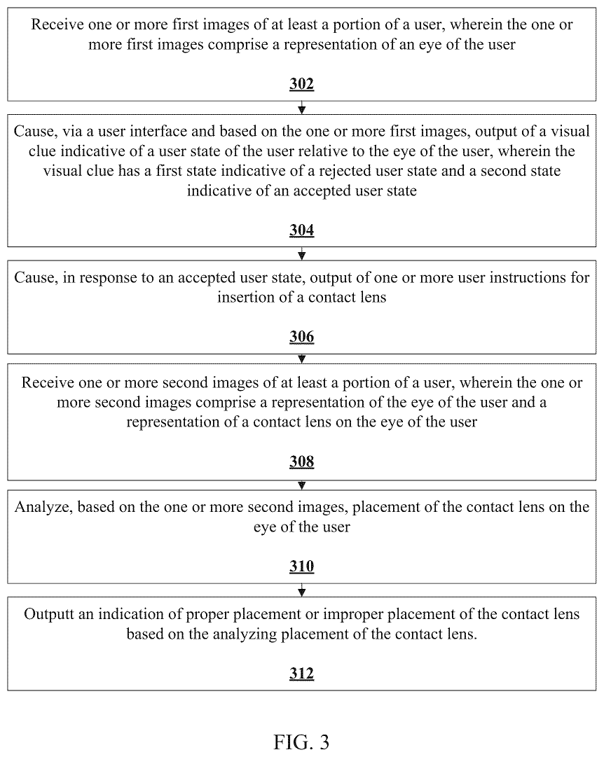

FIG. 3 depicts an example flowchart for a method for contact lens assistance such as insertion or removal of a contact lens from a user's eye(s). A system of one or more computers can be configured to perform particular operations or actions by virtue of having software, firmware, hardware, or a combination of them installed on the system that in operation causes or cause the system to perform the actions. As an example, a smartphone may be equipped to capture one or more images of a user (e.g., video, real-time video). As a further example, a user may implement the method on a device configured to display information to a user while capture “selfie” view images (e.g., video, real-time video) of the user.

At step 302, one or more first images of at least a portion of a user may be received. The one or more first images may comprise a representation of an eye of the user. The one or more first images may comprise a representation of an iris of the eye of the user. The one or more first images may be captured from a video. The one or more first images may be captured from a real-time video.

At step 304, a visual clue indicative of a user state of the user relative to the eye of the user may be caused to be outputted via a user interface and based on the one or more first images. The visual clue may have a first state indicative of a rejected user state and a second state indicative of an accepted user state. The user state may comprise one or more of an openness of the eye of the user, a characteristic of another eye of the user, and a finger placement of the user relative to the eye. Output prompts may be provided to the user via a user interface and may be based on the one or more first images until an accepted user state is detected. The user interface may be on any device or display. As an example, the user interface may be on a device configured to capture images of the face of the user while viewing the user interface.

At step 306, one or more user instructions for insertion of a contact lens may be caused to be outputted in response to an accepted user state. The one or more user instructions for insertion of a contact lens may comprise an audio instruction, a visual instruction, or both.

At step 308, one or more second images of at least a portion of a user may be received. The one or more second images may comprise a representation of the eye of the user and a representation of a contact lens on the eye of the user. The one or more second images may be captured from a video. The one or more second images may be captured from a real-time video.

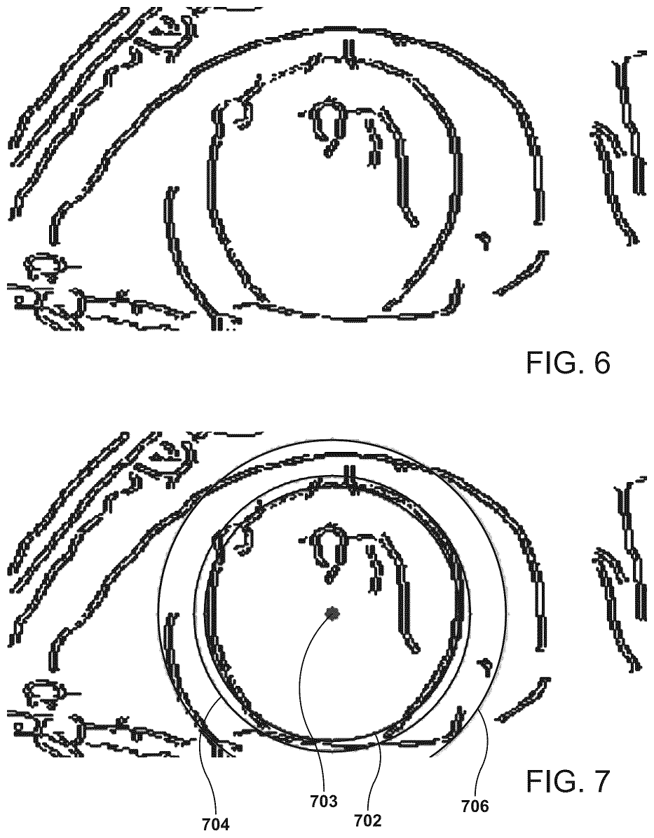

At step 310, placement of the contact lens on the eye of the user may be analyzed based on the one or more second images. Analyzing, based on the one or more second images, placement of the contact lens on the eye of the user may comprise using an edge detection model to detect an edge of the contact lens. Analyzing, based on the one or more second images, placement of the contact lens on the eye of the user may comprise pre-processing the one or more second images using one or more of cropping, masking, removal, or a combination thereof. Analyzing, based on the one or more second images, placement of the contact lens on the eye of the user may comprise pre-processing the one or more second images by digitally removing an artifact from the one or more second images. The artifact may comprise an eyelash of the user. Analyzing, based on the one or more second images, placement of the contact lens on the eye of the user may comprise using a mask for edge detection to detect an edge of the contact lens. As a non-limiting example, analysis of placement of the contact lens on the eye may comprises one or more processes described herein, for example implementing one or more processes described in reference to FIGS. 6-10. Other processes may be used.

At step 312, an indication of proper placement or improper placement of the contact lens may be outputted based on the analyzing placement of the contact lens.

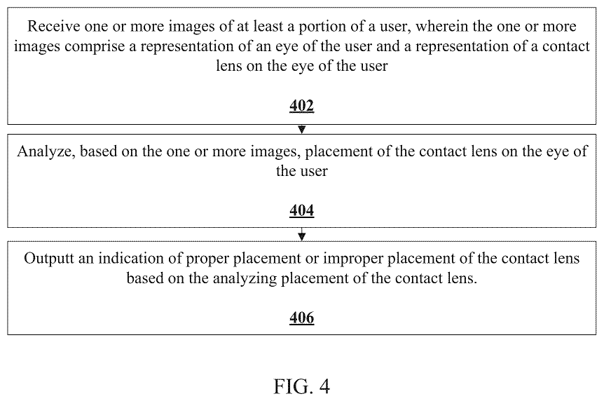

FIG. 4 depicts an example flowchart for a method for contact lens assistance such as insertion or removal of a contact lens from a user's eye(s). A system of one or more computers can be configured to perform particular operations or actions by virtue of having software, firmware, hardware, or a combination of them installed on the system that in operation causes or cause the system to perform the actions. As an example, a smartphone may be equipped to capture one or more images of a user (e.g., video, real-time video). As a further example, a user may implement the method on a device configured to display information to a user while capture “selfie” view images (e.g., video, real-time video) of the user.

At step 402, one or more images of at least a portion of a user may be received. The one or more images may comprise a representation of an eye of the user and a representation of a contact lens on the eye of the user. The one or more images may be captured from a video. The one or more images may be captured from a real-time video.

At step 404, placement of the contact lens on the eye of the user may be analyzed based on the one or more images. Analyzing, based on the one or more images, placement of the contact lens on the eye of the user may comprise using an edge detection model to detect an edge of the contact lens. Analyzing, based on the one or more images, placement of the contact lens on the eye of the user may comprise pre-processing the one or more images using one or more of cropping, masking, removal, or a combination thereof. Analyzing, based on the one or more images, placement of the contact lens on the eye of the user may comprise pre-processing the one or more images by digitally removing an artifact from the one or more second images. The artifact may comprise an eyelash of the user. Analyzing, based on the one or more images, placement of the contact lens on the eye of the user may comprise using a mask for edge detection to detect an edge of the contact lens. As a non-limiting example, analysis of placement of the contact lens on the eye may comprises one or more processes described herein, for example implementing one or more processes described in reference to FIGS. 6-10. Other processes may be used.

At step 406, an indication of proper placement or improper placement of the contact lens may be outputted based on the analyzing placement of the contact lens.

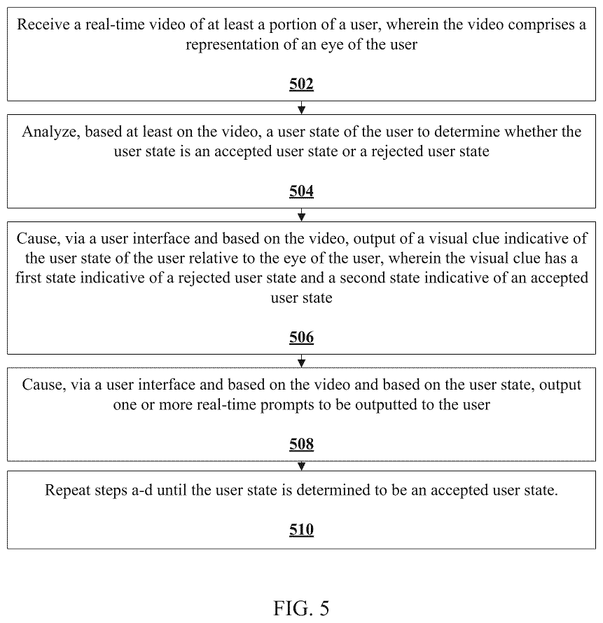

FIG. 5 depicts an example flowchart for a method for contact lens assistance such as insertion or removal of a contact lens from a user's eye(s). A system of one or more computers can be configured to perform particular operations or actions by virtue of having software, firmware, hardware, or a combination of them installed on the system that in operation causes or cause the system to perform the actions. As an example, a smartphone may be equipped to capture one or more images of a user (e.g., video, real-time video). As a further example, a user may implement the method on a device configured to display information to a user while capture “selfie” view images (e.g., video, real-time video) of the user.

At step 502, a real-time video of at least a portion of a user may be received. The video may comprise a representation of an eye of the user.

At step 504, a user state of the user may be analyzed based at least on the video to determine whether the user state is an accepted user state or a rejected user state. The accepted user state may comprise a properly inserted contact lens on the eye of the user. The accepted user state may comprise a properly removed contact lens on the eye of the user. The user state may comprise one or more of an openness of the eye of the user, a placement of a contact lens on the eye, a characteristic of another eye of the user, or a finger placement of the user relative to the eye, lens too high or lens too low. As a non-limiting example, analysis of placement of the contact lens on the eye may comprises one or more processes described herein, for example implementing one or more processes described in reference to FIGS. 6-10. Other processes may be used.

At step 506, a visual clue indicative of the user state of the user relative to the eye of the user may be caused to be output via a user interface and based on the video. The visual clue may have a first state indicative of a rejected user state and a second state indicative of an accepted user state. The user interface may be on any device or display. As an example, the user interface may be on a device configured to capture images of the face of the user while viewing the user interface.

At step 508, one or more real-time prompts may be caused to be outputted to the user via a user interface and based on the video and based on the user state. The user interface may be on any device or display. As an example, the user interface may be on a device configured to capture images of the face of the user while viewing the user interface. The one or more real-time prompts may comprise instruction for insertion or removal of a contact lens.

At step 510, steps 502-508 may be repeated until the user state is determined to be an accepted user state.

As described herein, analysis of a user state may comprise analyzing one or more of an openness of the eye of the user, a placement of a contact lens on the eye, a characteristic of another eye of the user, or a finger placement of the user relative to the eye. Such analysis may be based at least in part on images (or video) that capture a portion of the user such as the eye. Such images and the analysis of the same may benefit from certain processing techniques, such as those described herein below. Analyzing one or more images may comprise using an edge detection model to detect an edge of the contact lens. Analyzing one or more images may comprise pre-processing the one or more images using one or more of cropping, masking, removal, or a combination thereof. Analyzing one or more images may comprise pre-processing the one or more images by digitally removing an artifact from the one or more second images. The artifact may comprise an eyelash of the user. Analyzing one or more images may comprise using a mask for edge detection to detect an edge of the contact lens.

As a non-limiting example, analysis of user state (e.g., placement of the contact lens on the eye) may comprises one or more pre-processing methods where images or videos captured by devices disclosed herein may be pre-processed. Image processing methods may function before, during, or after additional method flows (see FIGS. 1-5) disclosed herein. Pre-processing methods include but are not limited to the variety of techniques described herein.

As non-limiting examples, image processing techniques may include tightly cropping images or videos of a user, highlighting portions of ocular structures of a user, adjusting modeling architectures, isolating blue channels, simplifying analysis methods and models, and other such strategies. Such strategies may be used to achieve or move toward one or more targets. These targets may be selected improve the overall function of method flows described herein. Targets include but are not limited to removing obstructions to ocular recognition, highlighting lens ridges of an ocular structure, finding a number of circular bodies related to an ocular structure, and the like. Such targets may assist in improved lens recognition. Improved lens recognition improves functions and outputs of additional method flows disclosed herein. Ocular structures may include those structures relating to eyes, corneas, irises, scleras, lenses, and other such vision-related bodies. As non-limiting examples, processing methods may occur after one or more images or videos of at least a portion of a user are captured by a device (e.g. steps 110, 302, 308, 402, 502, etc.) but before an output is provided to a user (e.g. steps 114, 312, 416, 508, etc.). As an additional non-limiting example, processing methods may occur during analysis steps (e.g. 310, 404, 504, etc.). As previously described, processing methods improve reliability and accuracy of outputs of method flows disclosed herein.

Processing may utilize a variety of methods to improve outputs to users. Processing may improve lens recognition, detection, and the like within one or more images or videos captured by a device. In a particular embodiment, processing may be used to recognize or detect a lens using topography of an ocular structure (e.g. an eye). In doing so, it can be said with a certain likelihood that a lens is found within an annular band with respect to an iris of an ocular structure. By identifying a characteristic dimension of an annular band with respect to an iris, one or more approximate characteristic dimensions of a lens may be discovered. These dimensions include but are not limited to minimum and maximum radii (e.g., lens edge) of a potential lens. These dimensions can allow the creation of a window of potential lens location.

A window may be defined by two or more concentric circles, though other windows are possible. As a non-limiting example, a window may be defined by two or more ellipses or other rounded structures. As a non-limiting example, an image or video of an ocular structure may include arcs or portions of a circle (e.g. an incomplete circle). Arcs may represent more than one ocular structure. As non-limiting examples, arcs may represent portions of an eyelid, portions of a lens, and the like. Processing methods disclosed herein may identify whether an arc is associated with a given ocular structure. A correct arc may be recognized as the arc associated with an ocular structure centered on an iris or within a tolerance of such center. As a non-limiting example, processing methods may differentiate between an arc representing an eyelid and an arc representing the lens. In such example, an arc representing an eyelid may not center on an iris while an arc representing a lens does. The creation of image windows using approximate characteristic dimensions improves the ability to accurately and reliably recognize one or more lenses within one or more images or videos captured by a device.

The present disclosure relates to methods of further improving accuracy and reliability processing methods. As a non-limiting example, improving qualities of an image or video of a user's ocular structure can improve accuracy and reliability of processing methods. Improved qualities include but are not limited to more accurate image or video capture of a user's iris. This includes capturing an image or video representing all existing boundaries of an iris. One such method is to increase the exposure area of a user's eye. Increasing the exposure area allows a device to more accurately capture an image or video of a user's ocular structures. More accurate image capture allows processing methods and other method flows disclosed herein to experienced improved function.

As an illustrative example, FIG. 6 illustrates an input image comprising at least a portion of a user's eye. This input image may be captured using various cameras and across various light spectrum. From the captured image, one or more boundaries (e.g., iris boundary, lens edge minimum boundary, lens edge maximum boundary, etc.) may be calculated (e.g., estimated). Various formulas and techniques may be used to calculate the one or more boundaries. As an example formula set one (1) may be used, as shown below:

Iris Boundary

rᵢᵣᵢₛ=11.7 mm,rₗₑₙₛ=14 mm,err=2 mm

Estimated Lens Circumference Minimum

rₗₑₙₛₘᵢₙ=(rₗₑₙₛ+rᵢᵣᵢₛ)/2≈10% more thanrᵢᵣᵢₛ,

Estimated Lens Circumference Maximum

rₗₑₙₛₘₐₓ=(rₗₑₙₛ+err)≈37% morethan rᵢᵣᵢₛ. (1)

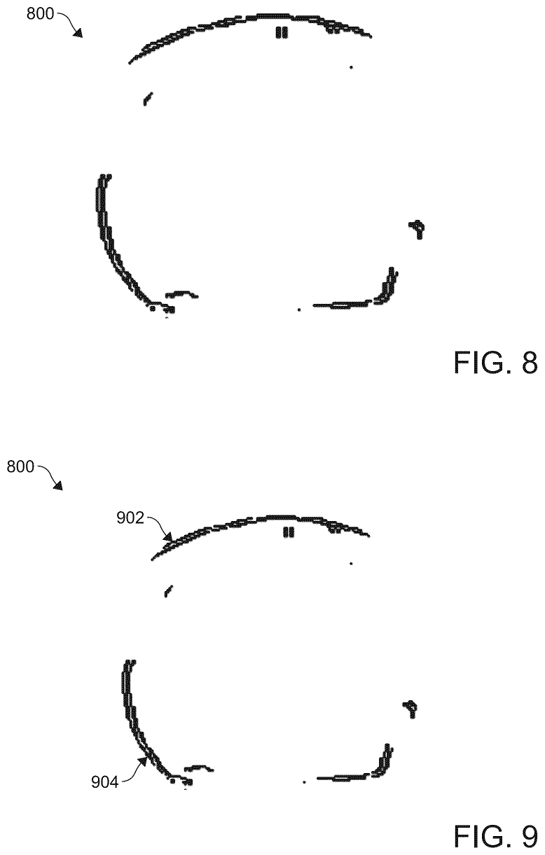

Other formulas, estimates, and representative values (e.g., 11.7 mm, 14 mm, 2 mm, etc.) may be used. FIG. 7 shows an illustrative example of calculated iris boundary 702 (and iris center 703), an estimated lens circumference minimum 704, and an estimated lens circumference maximum 706. Using these boundaries 702, 704, 706, a window may be calculated between the estimated lens circumference minimum 704 and the estimated lens circumference maximum 706. By removing (e.g., masking) the portions of the image outside of the calculated window, the image may be processed into a masked image 800, as shown in FIG. 8. This masked image represents a subset of the image information from the original image.

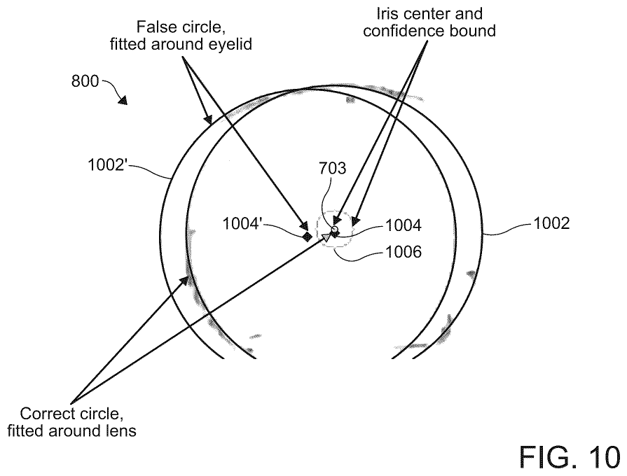

The masked image 800 may comprise one or more arcs or boundary indicators. Arcs may represent more than one ocular structure. As non-limiting examples, arcs may represent portions of an eyelid, portions of a lens, edges, boundaries, and/or the like. Such arcs may be analyzed and classified as a type such as an eyelid arc 902 or a lens boundary arc 904, as shown in FIG. 9. Types may be determined using formulas such as formula set one (1) or other methods to identify the arcs within a per-determined confidence level. For example, it may be determined that the located arcs form a portion of a representative circle. Estimating such circles, one may determine a center of the circle and may compare the center to the calculated center of the iris boundary. As an illustrative example, FIG. 10 illustrates the masked image 800 with an overlay of representative circles 1002, 1002′ and calculated circle centers 1004, 1004′ based on the arc geometry. Using a preset confidence boundary 1006, which may be derived from formula such as formula set one (1), it may be determined whether the representative circle center 1004, 1004′ is within the confidence boundary 1006 our outside the confidence boundary 1006. As a further example, a circle center 1004 within the confidence boundary 1006 may be identified as a correct circle that is fitted around the lens. A circle center 1004′ that is outside the confidence boundary 1006 may be identified as a false circle, fitted around something other than the target lens (e.g., an eyelid. The confidence boundary 1006 may be adjusted to control identification of arcs and artifacts in the masked image 800, which may be used to indicate proper or improper lens placement or lens removal.

Other lens detection algorithms may be used, for example, detection formulations published in Fully Automated Soft Contact Lens Detection from NIR Iris Images, by Kumar et al. Published in ICPRAM 24 Feb. 2016, Computer Science. As a further example, processing images may comprise using techniques (e.g., pre-processing techniques) such as those described in Using Iris and Sclera for Detection and Classification of Contact Lenses, but Gragnaniello et al., Pattern Recognition Letters, Published by Elsevier Online ISSN: 0167-8655. Additionally and alternatively, relevant structures of the face discussed herein, including the iris may be determined by the application of one or more combinations of deep learning, neural networks, and trained machine learning models, as will be appreciated by those skilled in the art. As one non-limiting example, convolutional neural networks as by ContlensNet: Robust Iris Contact Lens Detection Using Deep Convolutional Neural Networks, published in 2017 IEEE Winter Conference on Applications of Computer Vision (WACV), may be used to identify the iris.

In some examples, lidar data, for example as capable of being gathered by certain iPhone® models may be used in addition to or in lieu of video data to determine whether the user is performing actions leading toward a failure mode (e.g., improper or unsuccessful insertion or removal) or success (e.g., proper insertion or removal). Either or both of these data may be gathered either in a clinical or home environment and aggregated across a statistically sufficient number of users. Once gathered, the data is preferably ordered randomly and tagged in a database with tags associated with known failure modes or successes. The failure modes may include for example: i) an insufficient openness of the eye of the user, ii) an insufficient openness of another eye of the user; iii) an improper finger placement of the user relative to the eye; and iv) lens too high or low. This day may be then used to train a machine-learning model to recognize in real-time, a known failure mode or success by the user. The foreground-specialized teacher model may represent any suitable machine learning model that can be trained to detect and delineate objects in images or lidar data, such as a collection of neural network layers. This model may then be deployed in software on a user device, e.g., a mobile phone equipped with either or both video and lidar sensors, in order to to provide to the user auditory or visual feedback/prompts, including but not limited to visual cluses as described above, known to assist the user the user with successfully inserting the contact lens into the eye.

### EXAMPLES

Example 1: A method for contact lens assistance, the method comprising: causing, via a user interface, output of one or more lens type options; receiving, via the user interface, an indication of a select lens type of the one or more lens type options; causing, via the user interface, output of one or more lens operation options; receiving, via the user interface, an indication of a select lens operation of the one or more lens operation options; causing, via the user interface, output of one or more eye options; receiving, via the user interface, an indication of a select eye of the one or more eye options; causing, via the user interface and based upon one or more of the select lens type, the select lens operation, or the select eye, output of one or more user instructions for insert or removal of a contact lens, wherein the one or more user instructions incorporate a user feedback based on images of the user captured in real time.
Example 2: The method of example 1, wherein the lens type options comprise a representation of one or more of reusable or daily disposable.
Example 3: The method of any of examples 1-2, wherein the one or more lens operation options comprise a representation of one or more of insert or removal of a contact lens.
Example 4: The method of any of examples 1-3, wherein the one or more eye options comprise a representation of one or more of left eye or right eye.
Example 5: The method of any of examples 1-4, wherein the causing output of one or more user instructions for insert or removal of a contact lens is executed for the select eye and further comprising automatically causing, via the user interface and based upon one or more of the select lens type or the select lens operation, and for an unselected eye of the one or more eye options, output of one or more user instructions for insert or removal of a contact lens.
Example 6: The method of any of examples 1-5, wherein the one or more user instructions comprise wash and dry instructions.
Example 7: The method of any of examples 1-6, wherein the one or more user instructions comprise contact lens handling instructions.
Example 8: The method of any of examples 1-7, wherein the one or more user instructions comprise contact lens position instructions.
Example 9: The method of any of examples 1-8, wherein the one or more user instructions comprise contact lens orientation instructions.
Example 10: The method of any of examples 1-9, wherein the one or more user instructions comprise finger position instructions.
Example 11: The method of any of examples 1-10, wherein the one or more user instructions comprise camera configuration instructions.
Example 12: The method of any of examples 1-11, wherein the one or more user instructions comprise user feedback queries.
Example 13: A method for contact lens assistance, the method comprising: receiving one or more first images of at least a portion of a user, wherein the one or more first images comprise a representation of an eye of the user; causing, via a user interface and based on the one or more first images, output of a visual clue indicative of a user state of the user relative to the eye of the user, wherein the visual clue has a first state indicative of a rejected user state and a second state indicative of an accepted user state; causing, in response to an accepted user state, output of one or more user instructions for insertion of a contact lens; receiving one or more second images of at least a portion of a user, wherein the one or more second images comprise a representation of the eye of the user and a representation of a contact lens on the eye of the user; analyzing, based on the one or more second images, placement of the contact lens on the eye of the user; and outputting an indication of proper placement or improper placement of the contact lens based on the analyzing placement of the contact lens.
Example 14: The method of example 13, wherein the one or more first images comprise a representation of an iris of the eye of the user.
Example 15: The method of any of examples 13-14, wherein the user state comprises one or more of an openness of the eye of the user, a characteristic of another eye of the user, and a finger placement of the user relative to the eye.
Example 16: The method of any of examples 13-15, further comprising providing, via a user interface and based on the one or more first images, output prompts to the user until an accepted user state is detected.
Example 17: The method of any of examples 13-16, wherein the one or more user instructions for insertion of a contact lens comprise an audio instruction, a visual instruction, or both.
Example 18: The method of any of examples 13-17, wherein analyzing, based on the one or more second images, placement of the contact lens on the eye of the user comprises using an edge detection model to detect an edge of the contact lens.
Example 19: The method of any of examples 13-18, wherein analyzing, based on the one or more second images, placement of the contact lens on the eye of the user comprises pre-processing the one or more second images using one or more of cropping, masking, removal, or a combination thereof.
Example 20: The method of any of examples 13-19, wherein analyzing, based on the one or more second images, placement of the contact lens on the eye of the user comprises pre-processing the one or more second images by digitally removing an artifact from the one or more second images.
Example 21: The method of any of examples 13-20, wherein the artifact comprises an eyelash of the user.
Example 22: The method of any of examples 13-21, wherein analyzing, based on the one or more second images, placement of the contact lens on the eye of the user comprises using a mask for edge detection to detect an edge of the contact lens.
Example 23: The method of any of examples 13-22, wherein the one or more first images are captured from a video.
Example 24: The method of any of examples 13-23, wherein the one or more second images are captured from a video.
Example 25: The method of any of examples 13-24, wherein the one or more first images are captured from a real-time video.
Example 26: The method of any of examples 13-25, wherein the one or more second images are captured from a real-time video.
Example 27: A method for contact lens assistance, the method comprising: receiving one or more images of at least a portion of a user, wherein the one or more images comprise a representation of an eye of the user and a representation of a contact lens on the eye of the user; analyzing, based on the one or more images, placement of the contact lens on the eye of the user; and outputting an indication of proper placement or improper placement of the contact lens based on the analyzing placement of the contact lens.
Example 28: The method of example 27, wherein analyzing, based on the one or more images, placement of the contact lens on the eye of the user comprises using an edge detection model to detect an edge of the contact lens.
Example 29: The method of any of examples 27-28, wherein analyzing, based on the one or more images, placement of the contact lens on the eye of the user comprises pre-processing the one or more images using one or more of cropping, masking, removal, or a combination thereof.
Example 30: The method of any of examples 27-29, wherein analyzing, based on the one or more images, placement of the contact lens on the eye of the user comprises pre-processing the one or more images by digitally removing an artifact from the one or more second images.
Example 31: The method of any of examples 27-30, wherein the artifact comprises an eyelash of the user.
Example 32: The method of any of examples 27-31, wherein analyzing, based on the one or more images, placement of the contact lens on the eye of the user comprises using a mask for edge detection to detect an edge of the contact lens.
Example 33: The method of any of examples 27-32, wherein the one or more images are captured from a video.
Example 34: The method of any of examples 27-33, wherein the one or more images are captured from a real-time video.
Example 35: A method for contact lens assistance, the method comprising: a) receiving a real-time video of at least a portion of a user, wherein the video comprises a representation of an eye of the user; b) analyzing, based at least on the video, a user state of the user to determine whether the user state is an accepted user state or a rejected user state; c) causing, via a user interface and based on the video, output of a visual clue indicative of the user state of the user relative to the eye of the user, wherein the visual clue has a first state indicative of a rejected user state and a second state indicative of an accepted user state; d) causing, via a user interface and based on the video and based on the user state, output one or more real-time prompts to be outputted to the user; and repeating steps a-d until the user state is determined to be an accepted user state.
Example 36: The method of example 35, wherein the accepted user state comprise a properly inserted contact lens on the eye of the user.
Example 37: The method of any of examples 35-36, wherein the accepted user state comprise a properly removed contact lens on the eye of the user.
Example 38: The method of any of examples 35-37, wherein the one or more real-time prompts comprise instruction for insertion or removal of a contact lens.
Example 39: A method for contact lens assistance, the method comprising:
receiving a representation of at least a portion of a face of a user during an attempt by the user to insert a contact lens into an eye;
analyzing the representation to determine whether the user is in a known failure mode, wherein the analysis comprises applying a machine-learning model configured to recognize the known failure mode based on aggregated representations of users during the known failure mode;
providing, via a user interface, a real-time prompt known to assist the user with successfully inserting the contact lens into the eye.
Example 40: The method of example 39, wherein the representation comprises lidar data.
Example 40: The method of example 39, wherein the representation comprises video data.
Example 41: The method of example 39, wherein the failure mode includes one of: i) an insufficient openness of the eye of the user, ii) an insufficient openness of another eye of the user; iii) an improper finger placement of the user relative to the eye; and iv) lens too high or low.
Example 42: The method of example 39, wherein analyzing the machine-learning model is further configured to recognize a success mode and providing, via the user interface, positive feedback to the user.

Many operating systems, including Linux, UNIX®, OS/2®, and Windows®, iOS® and Android® are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be or comprise a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

From the above description, it can be seen that the present invention provides a system, computer program product, and method for the efficient execution of the described techniques. References in the claims to an element in the singular is not intended to mean “one and only” unless explicitly so stated, but rather “one or more.” All structural and functional equivalents to the elements of the above-described example embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are example only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising.”

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

What is claimed is:

1. A method for contact lens assistance, the method comprising:
causing, via a user interface, output of one or more lens type options;
receiving, via the user interface, an indication of a select lens type of the one or more lens type options;
causing, via the user interface, output of one or more lens operation options;
receiving, via the user interface, an indication of a select lens operation of the one or more lens operation options;
causing, via the user interface, output of one or more eye options;
receiving, via the user interface, an indication of a select eye of the one or more eye options;
causing, via the user interface and based upon one or more of the select lens type, the select lens operation, or the select eye, output of one or more user instructions for insert or removal of a contact lens,
wherein the one or more user instructions incorporate a user feedback based on images of the user captured in real time.

2. The method of claim 1, wherein the lens type options comprise a representation of one or more of reusable or daily disposable.

3. The method of claim 1, wherein the one or more lens operation options comprise a representation of one or more of insert or removal of a contact lens.

4. The method of claim 1, wherein the one or more eye options comprise a representation of one or more of left eye or right eye.

5. The method of claim 1, wherein the causing output of one or more user instructions for insert or removal of a contact lens is executed for the select eye and further comprising automatically causing, via the user interface and based upon one or more of the select lens type or the select lens operation, and for an unselected eye of the one or more eye options, output of one or more user instructions for insert or removal of a contact lens.

6. The method of claim 1, wherein the one or more user instructions comprise wash and dry instructions.

7. The method of claim 1, wherein the one or more user instructions comprise contact lens handling instructions.

8. The method of claim 1, wherein the one or more user instructions comprise contact lens position instructions.

9. The method of claim 1, wherein the one or more user instructions comprise contact lens orientation instructions.

10. The method of claim 1, wherein the one or more user instructions comprise finger position instructions.

11. The method of claim 1, wherein the one or more user instructions comprise camera configuration instructions.

12. The method of claim 1, wherein the one or more user instructions comprise user feedback queries.

13. A method for contact lens assistance, the method comprising:
receiving one or more first images of at least a portion of a user, wherein the one or more first images comprise a representation of an eye of the user;
causing, via a user interface and based on the one or more first images, output of a visual clue indicative of a user state of the user relative to the eye of the user, wherein the visual clue has a first state indicative of a rejected user state and a second state indicative of an accepted user state;
causing, in response to an accepted user state, output of one or more user instructions for insertion of a contact lens;
receiving one or more second images of at least a portion of a user, wherein the one or more second images comprise a representation of the eye of the user and a representation of a contact lens on the eye of the user;
analyzing, based on the one or more second images, placement of the contact lens on the eye of the user; and
outputting an indication of proper placement or improper placement of the contact lens based on the analyzing placement of the contact lens.

14. The method of claim 13, wherein the one or more first images comprise a representation of an iris of the eye of the user.

15. The method of claim 13, wherein the user state comprises one or more of an openness of the eye of the user, a characteristic of another eye of the user, and a finger placement of the user relative to the eye.

16. The method of claim 13, further comprising providing, via a user interface and based on the one or more first images, output prompts to the user until an accepted user state is detected.

17. The method of claim 13, wherein the one or more user instructions for insertion of a contact lens comprise an audio instruction, a visual instruction, or both.

18. The method of claim 13, wherein analyzing, based on the one or more second images, placement of the contact lens on the eye of the user comprises using an edge detection model to detect an edge of the contact lens.

19. The method of claim 13, wherein analyzing, based on the one or more second images, placement of the contact lens on the eye of the user comprises pre-processing the one or more second images using one or more of cropping, masking, removal, or a combination thereof.

20. The method of claim 13, wherein analyzing, based on the one or more second images, placement of the contact lens on the eye of the user comprises pre-processing the one or more second images by digitally removing an artifact from the one or more second images.

21. The method of claim 20, wherein the artifact comprises an eyelash of the user.

22. The method of claim 13, wherein analyzing, based on the one or more second images, placement of the contact lens on the eye of the user comprises using a mask for edge detection to detect an edge of the contact lens.

23. The method of claim 13, wherein the one or more first images are captured from a video.

24. The method of claim 13, wherein the one or more second images are captured from a video.

25. The method of claim 13, wherein the one or more first images are captured from a real-time video.

26. The method of claim 13, wherein the one or more second images are captured from a real-time video.

27. A method for contact lens assistance, the method comprising:
receiving one or more images of at least a portion of a user, wherein the one or more images comprise a representation of an eye of the user and a representation of a contact lens on the eye of the user;
analyzing, based on the one or more images, placement of the contact lens on the eye of the user; and
outputting an indication of proper placement or improper placement of the contact lens based on the analyzing placement of the contact lens.

28. The method of claim 27, wherein analyzing, based on the one or more images, placement of the contact lens on the eye of the user comprises using an edge detection model to detect an edge of the contact lens.

29. The method of claim 27, wherein analyzing, based on the one or more images, placement of the contact lens on the eye of the user comprises pre-processing the one or more images using one or more of cropping, masking, removal, or a combination thereof.

30. The method of claim 27, wherein analyzing, based on the one or more images, placement of the contact lens on the eye of the user comprises pre-processing the one or more images by digitally removing an artifact from the one or more second images.

31. The method of claim 30, wherein the artifact comprises an eyelash of the user.

32. The method of claim 27, wherein analyzing, based on the one or more images, placement of the contact lens on the eye of the user comprises using a mask for edge detection to detect an edge of the contact lens.

33. The method of claim 27, wherein the one or more images are captured from a video.

34. The method of claim 27, wherein the one or more images are captured from a real-time video.

35. A method for contact lens assistance, the method comprising:
a. receiving a real-time video of at least a portion of a user, wherein the video comprises a representation of an eye of the user;
b. analyzing, based at least on the video, a user state of the user to determine whether the user state is an accepted user state or a rejected user state;
c. causing, via a user interface and based on the video, output of a visual clue indicative of the user state of the user relative to the eye of the user, wherein the visual clue has a first state indicative of a rejected user state and a second state indicative of an accepted user state;
d. causing, via the user interface and based on the video and based on the user state, output one or more real-time prompts to be outputted to the user; and
e. repeating steps a-d until the user state is determined to be an accepted user state.

36. The method of claim 35, wherein the accepted user state comprise a properly inserted contact lens on the eye of the user.

37. The method of claim 35, wherein the accepted user state comprise a properly removed contact lens on the eye of the user.

38. The method of claim 35, wherein the one or more real-time prompts comprise instruction for insertion or removal of a contact lens.

39. A method for contact lens assistance, the method comprising:
a. receiving a representation of at least a portion of a face of a user during an attempt by the user to insert a contact lens into an eye;
b. analyzing the representation to determine whether the user is in a known failure mode, wherein the analysis comprises applying a machine-learning model configured to recognize the known failure mode based on aggregated representations of users during the known failure mode;
c. providing, via a user interface, a real-time prompt known to assist the user with successfully inserting the contact lens into the eye.

40. The method of claim 39, wherein the representation comprises lidar data.

41. The method of claim 39, wherein the representation comprises video data.

42. The method of claim 39, wherein the failure mode includes one of: i) an insufficient openness of the eye of the user, ii) an insufficient openness of another eye of the user; iii) an improper finger placement of the user relative to the eye; and iv) lens too high or low.

43. The method of claim 39, wherein analyzing the machine-learning model is further configured to recognize a success mode and providing, via the user interface, positive feedback to the user.
